(19) United States
(12) Patent Application Publication
(10) Pub. No.: US 2022/0235000 A1
(43) Pub. Date: Jul. 28, 2022

(54) Trisazo Compounds for Ink-jet Printing

(71) Applicant: LANXESS Deutschland GmbH, Cologne (DE)

(72) Inventors: Dominik Limper, Leverkusen (DE); Damien Moigno, Marly (CH)

(73) Assignee: LANXESS Deutschland GmbH, Cologne (DE)

(21) Appl. No.: 17/615,145

(22) PCT Filed: May 29, 2020
(86) PCT No.: PCT/EP2020/065033

§ 371 Date: Nov. 30, 2021

(30) Foreign Application Priority Data
Jun. 6, 2019 (EP) 19178667.2

(51) Int. Cl. C07C 309/74; C09D 11/328

(52) U.S. Cl. CPC C07C 309/74; C09D 11/328

(57) ABSTRACT

Novel trisazo compounds are disclosed. The compounds are well suited for the dyeing and printing of natural and synthetic materials, especially as recording liquids for inkjet printing.

The present invention relates to novel trisazo compounds and salts thereof, processes for the preparation thereof and the use thereof for dyeing and printing natural and synthetic materials, especially for use for inkjet printing.

A range of black dyes for application in inkjet printing processes are already known from the prior art.

In the field of industrial inkjet printing, for example, the dyes C.I. Acid Black 1 and C.I. Direct Black 19 are frequently employed.

In addition, EP-A 3 020 770 discloses black trisazo dyes for use in inkjet printing processes.

However, the dyes known from the prior art are still in need of improvement in some respects. In particular, the dyes known from the prior art do not possess sufficient storage stability, which manifests itself in a change in both the colour strength and the colour locus during storage of the liquid dye solutions.

There is therefore still the need for novel dyes for inkjet printing which overcome the abovementioned drawbacks.

The object of the present invention is therefore that of providing novel dyes for dyeing and printing natural and synthetic materials in black shades, especially for application for inkjet printing.

The present invention provides trisazo compounds of formula (I)

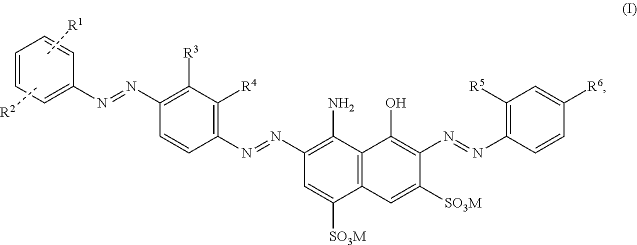

in which
R¹ is SO₃M or COOM, and
R² is hydrogen, SO₃M or COOM,
with the proviso that R¹ is SO₃M when R² is hydrogen, R³ and R⁴ independently of one another are hydrogen or CH₃,
R⁵ and R⁶ independently of one another are SO₃M, COOM or NO₂,
and
M is hydrogen, a monovalent metal cation, is ammonium or is alkylammonium which is mono- or polysubstituted identically or differently by C₁-C₄-alkyl.

Preference is given to trisazo compounds of formula (I) in which one of the radicals R⁵ or R⁶ is NO₂.

Examples of useful monovalent metal cations within the definition of M include sodium, potassium or lithium ions.

Useful examples of an alkylammonium which is mono- or polysubstituted identically or differently by C₁-C₄-alkyl include trimethylammonium, triethylammonium, triisopropylammonium, tributylammonium, preferably triethylammonium and triisopropylammonium.

The trisazo compounds of general formula (I) can as free acid or in the form of inorganic or organic salts.

They are preferably present as alkali metal or ammonium salt, especially as sodium salts.

M can be identical or different in the definitions SO₃M and COOM of the radicals R¹, R², R³, R⁵ and R⁶. Preferably, M has in each case the same definition in the definitions SO₃M and COOM of the radicals R¹, R², R³, R⁵ and R⁶.

Preference is given to trisazo compounds of formula (I)

in which
R¹ is SO₃M or COOM, and
R² is hydrogen, SO₃M or COOM,
with the proviso that R¹ is SO₃M when R² is hydrogen,
R³ and R⁴ independently of one another are hydrogen or CH₃, and
R⁵ and R⁶ independently of one another are SO₃M. COOM or NO₂,
with the proviso that one of the radicals R⁵ or R⁶ is NO₂,
and
M is hydrogen, sodium, potassium, lithium, ammonium or alkylammonium which is mono- or polysubstituted identically or differently by C₁-C₂-alkyl.

Particular preference is given to trisazo compounds of formula (I)

in which
R¹ is SO₃M or COOM, and
R² is hydrogen, SO₃M or COOM,
with the proviso that R¹ is SO₃M when R² is hydrogen, R³ and R⁴ independently of one another are hydrogen or CH₃, and
R⁵ and R⁶ independently of one another are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

Very particular preference is given to trisazo compounds of formula (I)

in which
R¹ is SO₃M or COOM, and
R² is hydrogen, SO₃M or COOM,
with the proviso that R¹ is SO₃M when R² is hydrogen, R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

Special preference is given to those compounds of formula (I) which correspond to formulae (la) or (Ib)

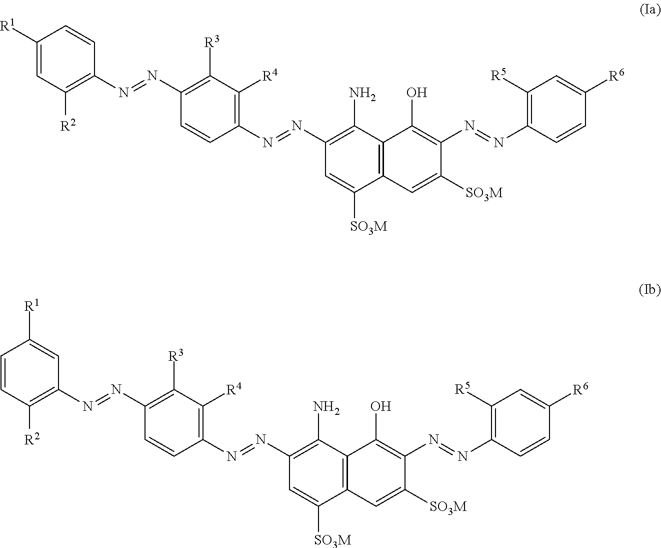

in which
R¹ is SO₃M or COOM, and
R² is hydrogen, SO₃M or COOM,
with the proviso that R¹ is SO₃M when R² is hydrogen,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

Special preference is given to compounds of the formula (I), (Ia) and (Ib),

in which
R¹ is SO₃M,
R² is SO₃M,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂, and
M is hydrogen, sodium, potassium, lithium, ammonium or triethylammonium.

Special preference is likewise given to compounds of the formula (I), (la) and (Ib),

in which

### R¹ is COOM

### R² is COOM,

R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium or triethylammonium.

The trisazo compounds of formula (I) according to the invention are outstandingly suitable for dyeing and printing various materials, especially in black shades.

The trisazo compounds of formula (I) according to the invention are suitable in particular for the dyeing and printing of cellulose-containing materials, in particular paper, cotton, linen and viscose, of animal hides and hair, in particular leather and wool, of eggshells and nanoporous materials and metals.

The trisazo compounds according to the invention can preferably be used for the bulk or surface colouration of paper. The dyes can also be used for the dyeing of yarns and piece goods made from cotton, viscose and linen in an exhaust process from a long liquor or in a continuous process.

The trisazo compounds according to the invention are especially suitable as dyes for aqueous and organic-solvent-based inks, in particular as recording fluids for inkjet printing and as dyes for writing devices such as for example pens and stamps. They are especially suitable for inkjet printing on porous, in particular nanoporous, recording sheets and on metals, paper and other cellulose-containing materials, and eggshells.

Nanoporous recording sheets are for example sheets made from nanoporous inorganic compounds, such as for example silicon dioxide, aluminium oxide/hydroxide, aluminium oxide or mixtures thereof.

The trisazo compounds according to the invention are suitable in particular as colourants for the production of liquid formulations for inkjet printing and for writing devices, or in the production of colour filters for optical and optoelectronic applications.

The colourings and prints obtained meet the highest quality demands. Surprisingly, the trisazo compounds of formula (I) according to the invention feature a markedly improved storage stability in aqueous solution. This makes it possible to store the aqueous solutions of the trisazo compounds according to the invention over relatively long periods of time, without the colour strength and colour locus of the solutions changing.

The present invention further provides formulations, in particular liquid formulations, containing at least one trisazo compound of formula (I), and the use of these formulations as dyeing composition for dyeing and printing applications, especially as recording fluids for inkjet printing and for writing implements.

The liquid formulation according to the invention generally contain 0.5% to 25% by weight, preferably 1.0% to 15% by weight and particularly preferably 2.0% to 8.0% by weight of at least one trisazo compound of formula (I), based on the overall formulation.

The formulation according to the invention are preferably water-based. In general they contain 40% to 99% by weight, preferably 70% to 95% by weight of water and optionally one or more of the following additives from the group of N-methyl-2-pyrrolidone, 2-pyrrolidone, 2-hexylpyrrolidone, hydroxyethylpyrrolidone, 2-propanol, ethanediol, hexane-1,2-diol, butane-1,2-diol, trimethylolpropane, diethylene glycol, diethylene glycol monobutyl ether, triethylene glycol monobutyl ether, dipropylene glycol monobutyl ether, glycerol, butyl lactate, urea, sulfolane, glycol ethers and biocides, where the total amount of all additives is from 0.01% to 50% by weight, preferably from 0.1% to 20% by weight, based on the overall formulation.

The liquid formulations according to the invention may also be based on organic solvents. In this case the formulations generally contain 40% to 99% by weight, preferably 70% to 95% by weight of at least one solvent from the group of N-methyl-2-pyrrolidone, 2-pyrrolidone, 2-hexylpyrrolidone, hydroxyethylpyrrolidone, 2-propanol, ethanediol, hexane-1,2-diol, butane-1,2-diol, trimethylolpropane, diethylene glycol, diethylene glycol monobutyl ether, triethylene glycol monobutyl ether, dipropylene glycol monobutyl ether, glycerol, butyl lactate and optionally one or more of the following additives from the group of urea, sulfolane, glycol ethers, biocides, where the total amount of all additives is from 0.01% to 50% by weight, preferably from 0.1% to 20% by weight, based on the overall formulation.

The formulations according to the invention can be produced by mixing at least one trisazo compound of formula (I) according to the invention with water and/or with at least one organic solvent and optionally with one or more additives.

The present invention further provides for the use of a formulation according to the invention as a dyeing composition, in particular ink, recording fluid or textile colourant.

The trisazo compounds of formula (I) according to the invention can be prepared by reacting at least one compound of formula (II)

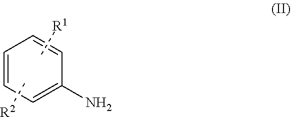

in which
R¹ and R² have the definitions specified for formula (I),
with a diazotization reagent,
and subsequently reacting the reaction mixture with at least one compound of formula (III)

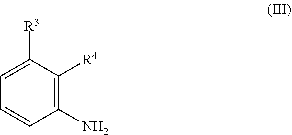

in which
R³ and R⁴ have the definitions specified for formula (I),
to give the intermediate of formula (IV)

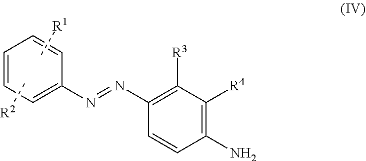

in which
R¹, R², R³ and R⁴ have the definitions specified for formula (I), and further reacting the intermediate of formula (IV) with a diazotization reagent, and subsequently reacting the reaction mixture thus obtained with a compound of formula (V)

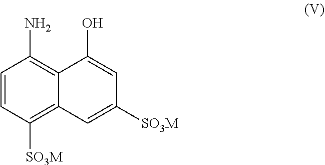

to form the intermediate of formula (VI)

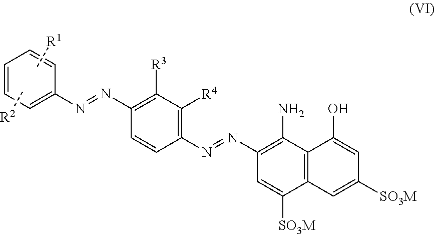

in which
R¹, R², R³ and R⁴ have the definitions specified for formula (I),
subsequently reacting the intermediate of formula (VI) with the reaction product obtainable by reacting a compound of formula (VII)

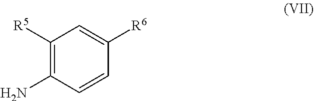

in which R⁵ and R⁶ have the definitions specified for formula (I), with a diazotization reagent.

As diazotization reagent, inorganic and organic nitrites, nitrosylsulfuric acid, preferably sodium nitrite or methyl nitrite, are suitable.

The diazotization steps of the process according to the invention are preferably performed in the manner known to those skilled in the art. The pH for the diazotization steps can be varied within a wide range. The pH can typically be in the range from 0 to 12.

The diazotization reagent can be used individually or in any desired mixture with one another.

The preparation process according to the invention is typically conducted in a temperature range of from −20° C. to +90° C., preferably from −10° C. to +60° C.

Expediently, the individual steps of the process according to the invention are performed at ambient pressure, but the reaction may also be conducted in the range from 1000 to 10 000 hPa, preferably 10 to 5000 hPa. Ambient pressure is understood to mean an air pressure in the range from about 925 hPa to 1070 hPa.

The trisazo compounds according to the invention are usually not isolated, but rather further used directly in the aqueous solution formed. Purification of the product solution, for example by using ion exchangers or pressure permeation, is possible but not absolutely necessary. For the workup and isolation of the trisazo dyes according to the invention, it is, however, possible to precipitate them with ethanol and to wash them on a suction filter.

The examples which follow are intended to illustrate the present invention but without restricting it thereto.

### EXAMPLES

### Example A

Example A is the dye C.I. Acid Black 1 (not according to the invention) corresponding to the formula

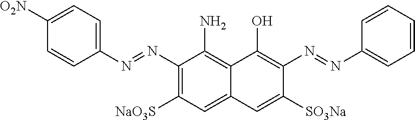

Product from TCI Chemicals.

### Example B

Example B is the dye C.I. Direct Black 19 (not according to the invention) corresponding to the formula

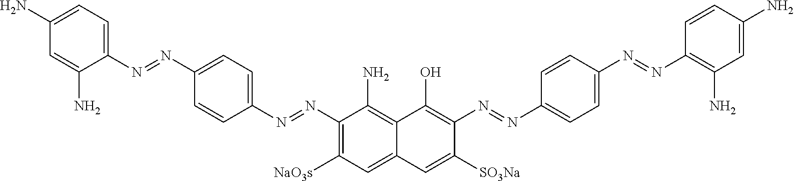

Product from Dystar under the trade name Jettex Direct Black 19.

### Example 1

1.00 mmol of 5-aminoisophthalic acid were suspended in 80 equivalents of water. Next, 2.50 equivalents of hydrochloric acid in the form of a 37% aqueous solution were added and the reaction mixture was cooled down to 2° C. A 40% aqueous sodium nitrite solution was added in an equimolar amount and the mixture was stirred for 1 hour at 3° C. The nitrite excess was removed by adding amidosulfonic acid. An equimolar amount of m-toluidine was subsequently suspended in water and added to the reaction mixture within 60 minutes. Subsequently, the pH of the reaction mixture was adjusted using hydrochloric acid to pH 1.8 and the mixture was stirred for 30 minutes. The black precipitate was isolated by filtration and washed with aqueous hydrochloric acid (1% aqueous HCl solution).

The filter cake was suspended in 80 equivalents of water and the suspension was stirred for 40 minutes at 4° C. 2.75 equivalents of HCl were subsequently added and an equimolar amount of a 40% aqueous sodium nitrite solution was then metered in over a period of 60 minutes. The nitrite excess was removed by adding amidosulfonic acid. Next, an equimolar amount of 4-amino-5-hydroxynaphthalene-1,7-disulfonic acid was added and the suspension was stirred for 2 hours. The suspension was warmed to 15° C. and the pH adjusted to 1.1 by adding aqueous HCl solution. The reaction mixture was subsequently stirred for 12 hours and the pH set to 7.7 using an aqueous NaOH solution (suspension 1). An equimolar amount of 4-[(4-amino-3-nitrophenyl)amino]-4-oxobutanoic acid was suspended in 70 equivalents of water and 4 equivalents of hydrochloric acid were added. An equimolar amount of an aqueous sodium nitrite solution was then added. The nitrite excess was removed by adding amidosulfonic acid. Suspension 1 was metered into this suspension at a temperature of 3° C. The pH was adjusted to 8.8 using an aqueous NaOH solution. Subsequently, the reaction mixture was warmed to room temperature. For further purification, filtering was performed over an SiO₂ bed (Celite®).

0.42 mmol of the dye of the formula

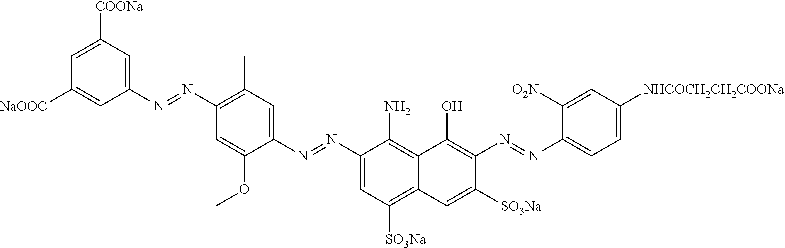

was obtained. Yield: 42%

### Example 2

The dye of example 2 was prepared analogously to example 1, with the difference that instead of 5-aminoisophthalic acid an equimolar amounts of p-aminobenzoic acid were used. 0.44 mmol of the dye of the formula

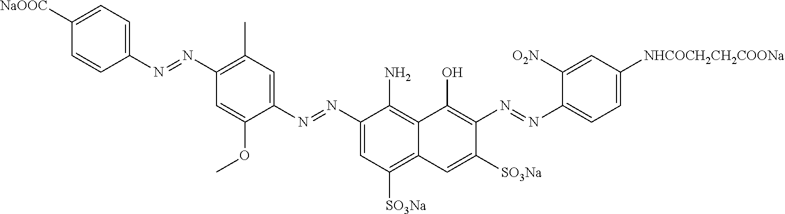

was obtained. Yield: 44%

### Example 3

The dye of example 3 was prepared analogously to example 1, with the difference that instead of 5-aminoisophthalic acid an equimolar amount of aniline-2,4-disulfonic acid was used and instead of 4-[(4-amino-3-nitrophenyl)amino]-4-oxobutanoic acid an equimolar amount of 4-nitroaniline-2-sulfonic acid was used. 0.49 mmol of the dye of the formula

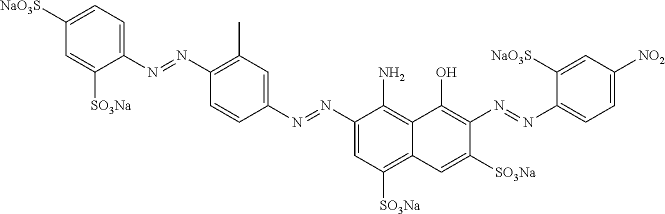

was obtained. Yield 49%

### Example 4

The dye of example 4 was prepared analogously to example 1, with the difference that instead of 5-aminoisophthalic acid an equimolar amount of aniline-2,4-disulfonic acid was used and instead of 4-[(4-amino-3-nitrophenyl)amino]-4-oxobutanoic acid an equimolar amount of 2-nitroaniline-4-sulfonic acid was used. 0.50 mmol of the dye of the formula

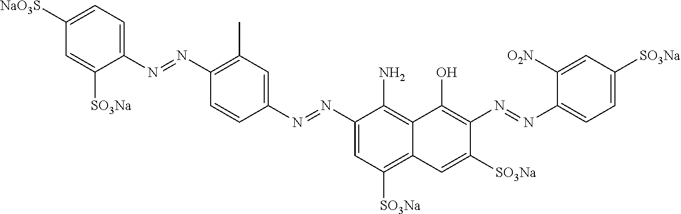

was obtained. Yield 50%

### Example 5

The dye of example 5 was prepared analogously to example 1, with the difference that instead of 5-aminoisophthalic acid an equimolar amount of aniline-2,4-disulfonic acid was used, instead of 4-[(4-amino-3-nitrophenyl)amino]-4-oxobutanoic acid an equimolar amount of 2-nitroaniline-4-sulfonic acid was used and instead of m-toluidine an equimolar amount of o-toluidine was used. 0.53 mmol of the dye of the formula

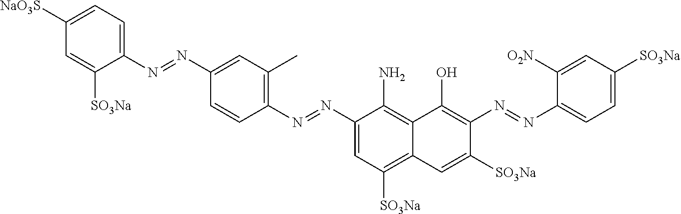

was obtained. Yield 53%

### Example 6

The dye of example 6 was prepared analogously to example 1, with the difference that instead of 5-aminoisophthalic acid an equimolar amount of aniline-2,4-disulfonic acid was used, instead of 4-[(4-amino-3-nitrophenyl)amino]-4-oxobutanoic acid an equimolar amount of 4-nitroaniline-2-sulfonic acid was used and instead of m-toluidine an equimolar amount of o-toluidine was used. 0.48 mmol of the dye of the formula

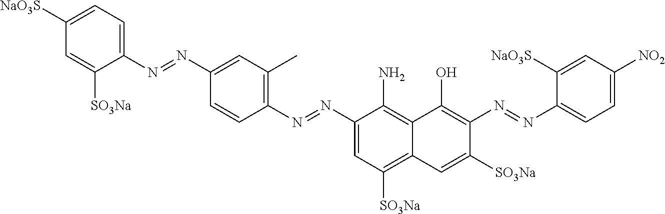

was obtained. Yield 48%

### Example 7

The dye of example 7 was prepared analogously to example 1, with the difference that instead of 5-aminoisophthalic acid an equimolar amount of aniline-2,5-disulfonic acid was used and instead of 4-[(4-amino-3-nitrophenyl)amino]-4-oxobutanoic acid an equimolar amount of 4-nitroaniline-2-sulfonic acid was used. 0.42 mmol of the dye of the formula

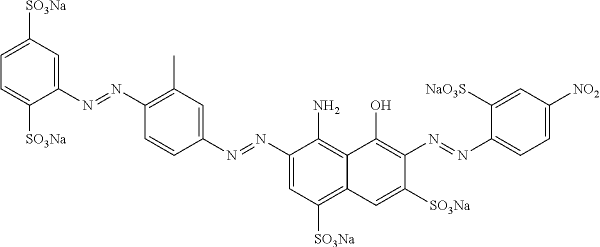

was obtained. Yield 42%

### Example 8

The dye of example 8 was prepared analogously to example 1, with the difference that instead of 5-aminoisophthalic acid an equimolar amount of aniline-2,5-disulfonic acid was used and instead of 4-[(4-amino-3-nitrophenyl)amino]-4-oxobutanoic acid an equimolar amount of 2-nitroaniline-4-sulfonic acid was used. 0.49 mmol of the dye of the formula

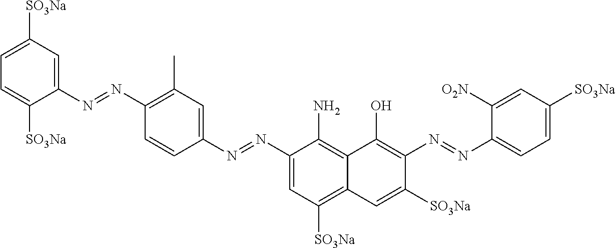

was obtained. Yield 49%

### Example 9

The dye of example 9 was prepared analogously to example 1, with the difference that instead of 5-aminoisophthalic acid an equimolar amount of aniline-2,5-disulfonic acid was used, instead of 4-[(4-amino-3-nitrophenyl)amino]-4-oxobutanoic acid an equimolar amount of 2-nitroaniline-4-sulfonic acid was used and instead of m-toluidine an equimolar amount of o-toluidine was used. 0.53 mmol of the dye of the formula

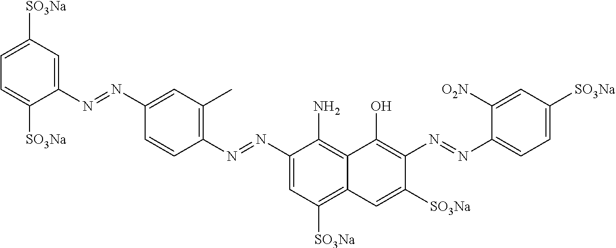

was obtained. Yield 53%

### Example 10

The dye of example 10 was prepared analogously to example 1, with the difference that instead of 5-aminoisophthalic acid an equimolar amount of aniline-2,5-disulfonic acid was used, instead of 4-[(4-amino-3-nitrophenyl)amino]-4-oxobutanoic acid an equimolar amount of 4-nitroaniline-2-sulfonic acid was used and instead of m-toluidine an equimolar amount of o-toluidine was used. 0.50 mmol of the dye of the formula

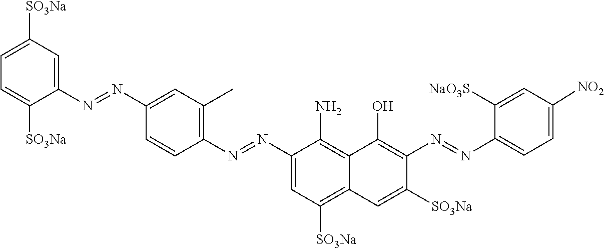

was obtained. Yield 50%

### Example 11

The dye of example 11 was prepared analogously to example 1, with the difference that instead of 5-aminoisophthalic acid an equimolar amount of aniline-2,5-disulfonic acid was used, instead of 4-[(4-amino-3-nitrophenyl)amino]-4-oxobutanoic acid an equimolar amount of 4-nitroaniline-2-sulfonic acid was used and instead of m-toluidine an equimolar amount of o-toluidine was used. Following the synthesis, 10.0 equivalents of ammonium chloride were added and the reaction mixture was stirred for 2 h at room temperature. For further purification, filtering was performed over an SiO₂ bed (Celite®). 0.49 mmol of the dye of the formula

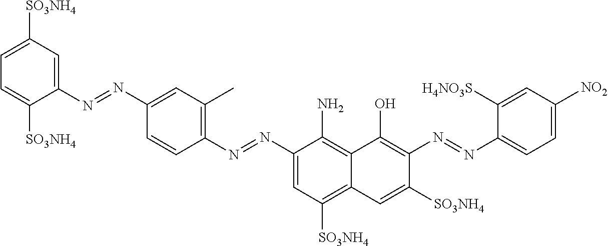

was obtained. Yield 49%

### Example 12

The dye of example 11 was prepared analogously to example 1, with the difference that instead of 5-aminoisophthalic acid an equimolar amount of aniline-2,5-disulfonic acid was used, instead of 4-[(4-amino-3-nitrophenyl)amino]-4-oxobutanoic acid an equimolar amount of 4-nitroaniline-2-sulfonic acid was used and instead of m-toluidine an equimolar amount of o-toluidine was used. Following the synthesis, 10.0 equivalents of triethylamine were added and the reaction mixture was stirred for 2 h at room temperature. For further purification, filtering was performed over an SiO₂ bed (Celite®). 0.47 mmol of the dye of the formula

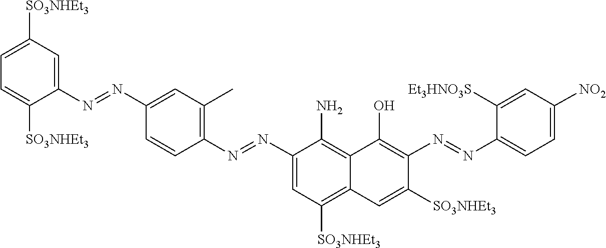

was obtained. Yield 47%

### Example 13

The dye of example 13 was prepared analogously to example 1, with the difference that instead of 5-aminoisophthalic acid an equimolar amount of 4-aminophthalic acid was used and instead of 4-[(4-amino-3-nitrophenyl)amino]-4-oxobutanoic acid an equimolar amount of 2-amino-4-nitrobenzoic acid was used. 0.50 mmol of the dye of the formula

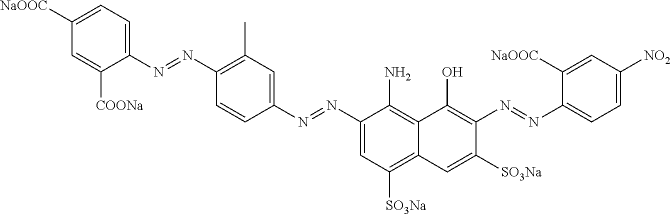

was obtained. Yield 50%

### Example 14

The dye of example 13 was prepared analogously to example 1, with the difference that instead of 5-aminoisophthalic acid an equimolar amount of 4-aminophthalic acid was used and instead of 4-[(4-amino-3-nitrophenyl)amino]-4-oxobutanoic acid an equimolar amount of 4-nitroaniline-2-sulfonic acid was used. 0.52 mmol of the dye of the formula

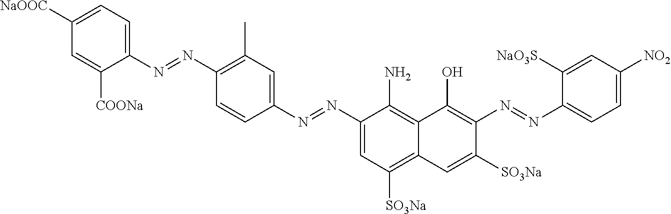

was obtained. Yield 52%
Determining the storage stability of an aqueous dye solution:

The E1/1 value specified is a hypothetical absorbance value which would be obtained if a 1 percent by weight solution of the respective compound (dissolved in water) were to be measured in a cuvette with a 1 cm path length.

The change in the E1/1 value, and the change in λₘₐₓ, serve as a measure for the storage stability of an aqueous dye solution. For the determination of the storage stability of the dyes according to the invention and not according to the invention of examples A and B and 1 to 14, aqueous dye solutions having a concentration of 19% by weight were in each case produced and these dye solutions were stored in a closed glass vessel for 28 days at 45° C. The E1/1 value was determined in each case after 0 days and after 28 days in accordance with the procedure described above. The change in the E1/1 value over this period amounts to the value ΔE1/1=E1/1 (28 days)−E1/1 (0 days). The change in Amax over this period amounts to the value Δλₘₐₓ=λₘₐₓ (28 days)−λₘₐₓ (0 days).

Here, the change in the E1/1 value is a measure for the loss of colour strength over the storage period, the change in Amax is a measure for the shift of the colour locus over the storage period.

The results are reproduced in table 1.

TABLE 1

| | according to the | λₘₐₓ | | Δ λₘₐₓ |
|---|---|---|---|---|
| Example | invention | [nm] | Δ E1/1 at λₘₐₓ | [nm] |
| A | no | 619 | −14 | 5 |
| B | no | 618 | −11 | −6 |
| 1 | no | 621 | −7 | −8 |
| 2 | no | 622 | −9 | −5 |
| 3 | yes | 596 | <−1 | 0 |
| 4 | yes | 615 | −3 | −1 |
| 5 | yes | 616 | <−1 | 1 |
| 6 | yes | 595 | −3 | 0 |
| 7 | yes | 596 | <−1 | 1 |
| 8 | yes | 613 | −3 | −1 |
| 9 | yes | 613 | −2 | 1 |
| 10 | yes | 598 | −3 | 1 |
| 11 | yes | 598 | −2 | −1 |
| 12 | yes | 600 | −2 | 0 |
| 13 | yes | 625 | −3 | 1 |
| 14 | yes | 619 | <−1 | −1 |

Conclusion: As can be seen from table 1, the aqueous solutions of the trisazo compounds according to the invention of examples 3 to 14 display a markedly smaller change in the absorbance values and in Amax compared to the dyes not according to the invention of examples A and B and 1 and 2 of the prior art, even after relatively long storage over a period of 28 days at an elevated temperature of 45° C. This means that for the aqueous solutions of the trisazo compounds according to the invention the colour strength and colour locus remain virtually unchanged over a period of 28 days, whereas in contrast for the dyes not according to the invention marked deviations can be detected.

1. A trisazo compound of formula (I) 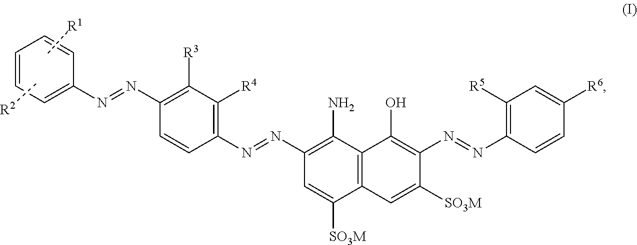
in which
R¹ is SO₃M or COOM, and
R² is hydrogen, SO₃M or COOM,
with the proviso that R¹ is SO₃M when R² is hydrogen, R³ and R⁴ independently of one another are hydrogen or CH₃,
R⁵ and R⁶ independently of one another are SO₃M, COOM or NO₂, and
M is hydrogen, a monovalent metal cation, ammonium or alkylammonium which is mono- or polysubstituted identically or differently by C₁-C₄-alkyl.

2. The trisazo compound according to claim 1, wherein
R¹ is SO₃M or COOM,
and
R² is hydrogen, SO₃M or COOM,
with the proviso that R¹ is SO₃M when R² is hydrogen,
R³ and R⁴ independently of one another are hydrogen or CH₃,
and
R⁵ and R⁶ independently of one another are SO₃M, COOM or NO₂, with the proviso that one of the radicals R⁵ or R⁶ is NO₂,
and
M is hydrogen, sodium, potassium, lithium, ammonium or alkylammonium which is mono- or polysubstituted identically or differently by C₁-C₂-alkyl.

3. The trisazo compound according to claim 1, wherein
R1 is SO₃M or COOM,
and
R² is hydrogen, SO₃M or COOM,
with the proviso that R¹ is SO₃M when R² is hydrogen, R³ and R⁴ independently of one another are hydrogen or CH₃,
and
R⁵ and R⁶ independently of one another are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

4. The trisazo compound according to claim 1, wherein
R¹ is SO₃M or COOM,
and
R² is hydrogen, SO₃M or COOM,
with the proviso that R¹ is SO₃M when R² is hydrogen,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

5. The trisazo compound according to claim 1, wherein the compound is of formula (Ia) or (Ib) 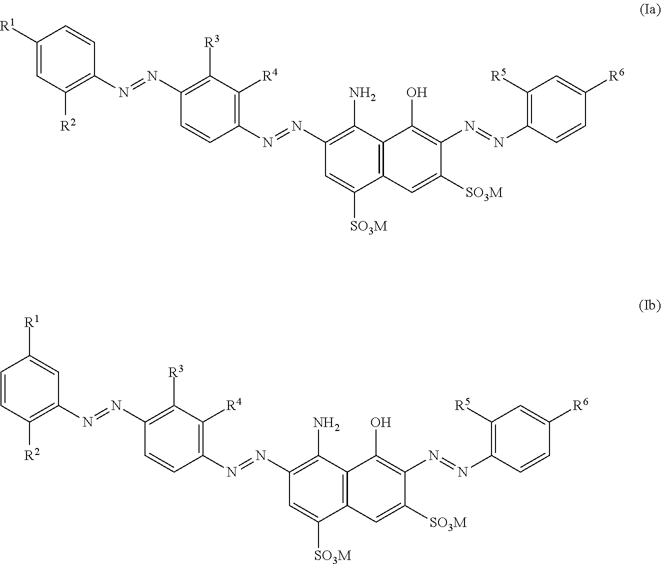
in which
R¹ is SO₃M or COOM,
and
R² is hydrogen, SO₃M or COOM,
with the proviso that R¹ is SO₃M when R² is hydrogen,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium, trimethylammonium or triethylammonium.

6. The trisazo compound according to claim 1, wherein
R¹ is SO₃M,
R² is SO₃M,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂, and
M is hydrogen, sodium, potassium, lithium, ammonium or triethylammonium.

7. The trisazo compound according to claim 1, wherein
R¹ is COOM,
R² is COOM,
R³ and R⁴ are hydrogen or CH₃,
with the proviso that R³ and R⁴ are not both simultaneously hydrogen and are not both simultaneously CH₃,
R⁵ and R⁶ are SO₃M, COOM or NO₂,
with the proviso that one of the radicals R⁵ or R⁶ is NO₂,
and
M is hydrogen, sodium, potassium, lithium, ammonium or triethylammonium.

8. The trisazo compound according to claim 1, wherein the compound is of the formula

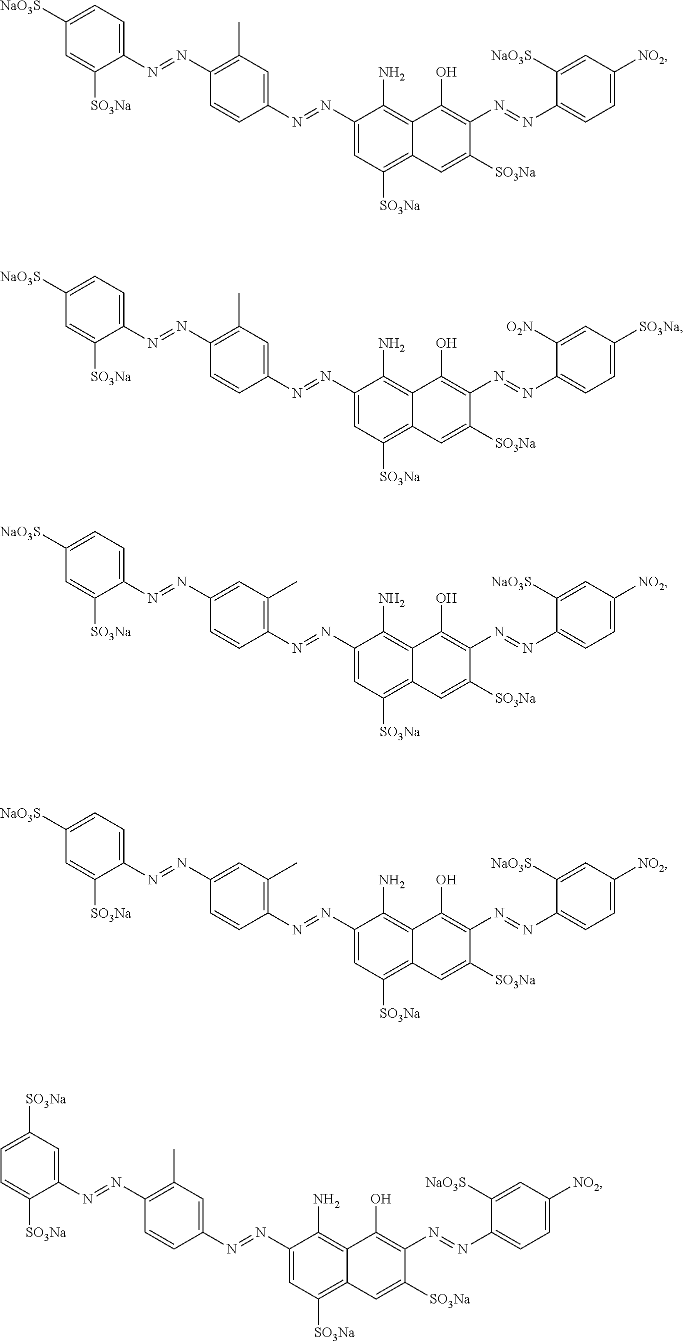

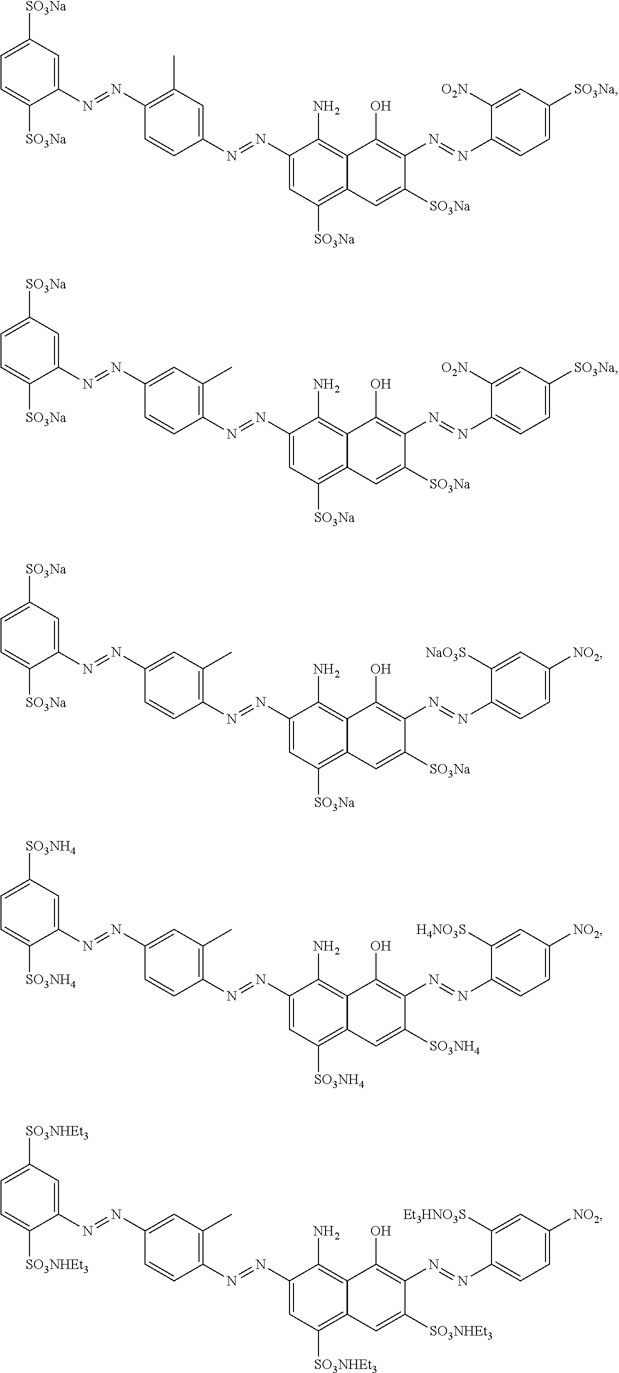

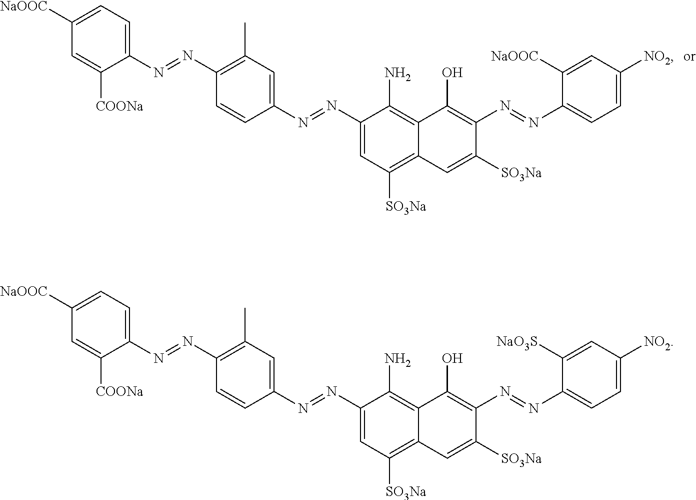

9. A process for the dyeing and printing of cellulose-containing materials, animal hides, animal hair, eggshells, porous materials, or metals, comprising applying a dye containing at least one trisazo compound according to claim 1.

10. A formulation containing at least one trisazo compound according to claim 1.

11. The formulation according to claim 10, wherein the formulation is a liquid.

12. The formulation according to claim 10, containing 0.5% to 25% by weight of at least one trisazo compound according to claim 1, based on the overall formulation.

13. The formulation according to claim 10, containing 40% to 99% by weight of water and optionally one or more additives from the group of N-methyl-2-pyrrolidone, 2-pyrrolidone, 2-hexylpyrrolidone, hydroxyethylpyrrolidone, 2-propanol, ethanediol, hexane-1,2-diol, butane-1,2-diol, trimethylolpropane, diethylene glycol, diethylene glycol monobutyl ether, triethylene glycol monobutyl ether, dipropylene glycol monobutyl ether, glycerol, butyl lactate, urea, sulfolane, glycol ethers and biocides in a total amount of from 0.01% to 50% by weight, based on the overall formulation.

14. The formulation according to claim 10, containing 40% to 99% by weight of at least one solvent from the group of N-methyl-2-pyrrolidone, 2-pyrrolidone, 2-hexylpyrrolidone, hydroxyethylpyrrolidone, 2-propanol, ethanediol, hexane-1,2-diol, butane-1,2-diol, trimethylolpropane, diethylene glycol, diethylene glycol monobutyl ether, triethylene glycol monobutyl ether, dipropylene glycol monobutyl ether, glycerol, butyl lactate and optionally one or more additives from the group of urea, sulfolane, glycol ethers, biocides in a total amount of from 0.01% to 50% by weight, based on the overall formulation.

15. (canceled)

16. A process for preparing trisazo compounds according to claim 1, comprising reacting at least one compound of formula (II) 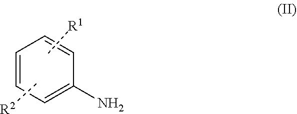
in which
R¹ and R² have the definitions specified in claim 1,
with a diazotization reagent, and reacting the reaction mixture thus obtained with at least one compound of formula (III) 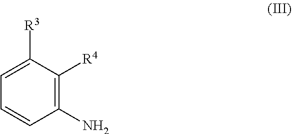
in which
R³ and R⁴ have the definitions specified in claim 1,
to give the intermediate of formula (IV) 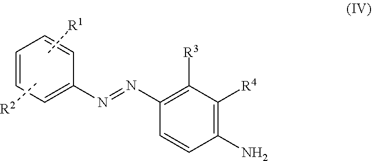
in which
R¹, R², R³ and R⁴ have the definitions specified in claim 1,
and reacting the intermediate of formula (IV) with a diazotization reagent, and reacting the reaction mixture thus obtained with a compound of formula (V) 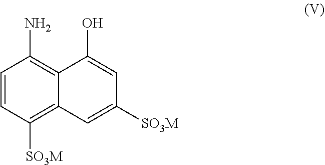
to give the intermediate of formula (VI) 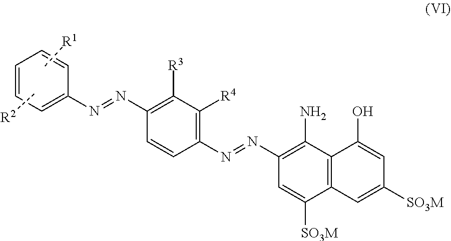
in which
R¹, R², R³ and R⁴ have the definitions specified in claim 1, and reacting the intermediate of formula (VI) with a reaction product obtainable by reacting a compound of formula (VII) 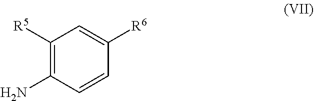
in which
R⁵ and R⁶ have the definitions specified in claim 1,
with a diazotization reagent, to form the trisazo compound of formula (I), and isolating the trisazo compound by filtration of the reaction mixture.
